SEQUENCE LISTING (2 sequences)

SEQ ID NO: 1 - DNA, 23 bp, Artificial
  siRNA
ggcagacuuuagaugcuucuutt

SEQ ID NO: 2 - DNA, 23 bp, Artificial
  siRNA
ucuuccaucaaugacagaccctt

(19) United States
(12) Patent Application Publication
(10) Pub. No.: US 2016/0040126 A1
(43) Pub. Date: Feb. 11, 2016

(54) REGULATION OF DIFFERENTIATION INTO DOPAMINERGIC NEURONS BY METALLOPROTEASE

(71) Applicant: Korea University Research and Business Foundation, Seoul (KR)

(72) Inventors: Ja-Hyun Baik, Seoul (KR); Se Hyoun Yoon, Seoul (KR)

(21) Appl. No.: 14/379,781

(22) PCT Filed: Feb. 21, 2013
(86) PCT No.: PCT/KR13/01389

§ 371 Date: Aug. 20, 2014

(30) Foreign Application Priority Data
Feb. 21, 2012 (KR) 10-2012-0017667
Feb. 21, 2012 (KR) 10-2012-0017668

(51) Int. Cl. C12N 5/0793

(52) U.S. Cl. CPC C12N 5/0619; C12N 2506/08; C12N 2501/734; C12N 2503/02

(57) ABSTRACT

The present invention relates to a method for regulating the differentiation of neural stem cells or neural progenitor cells into dopaminergic neural cells, the method comprising increasing or inhibiting the activity of ADAM17 and/or ADAM10 in neural stem cells or neural progenitor cells, and to the use of an activator or inhibitor of ADMA17 and/or ADAM10.

The method and composition according to the invention can regulate the activity of ADAM17 and/or ADAM10 in neural stem cells or neural progenitor cells to increase dopaminergic neural cells in the midbrain area, and thereby providing the effect of treating diseases induced by the death of dopaminergic neural cells such as Parkinson's disease. In addition, the method and composition according to the invention can inhibit the activity of ADAM17 and/or ADAM10, and thereby improving effect of treating diseases such as tumor. Thus, the present invention is very useful.

### TECHNICAL FIELD

The present invention relates to a method for regulating the differentiation of neural stem cells or neural progenitor cells into dopaminergic neural cells, the method comprising increasing or inhibiting the activity of ADAM17 and/or ADAM10 in neural stem cells or neural progenitor cells, and to the use of an activator or inhibitor of ADAM17 and/or ADAM10.

### BACKGROUND ART

It is known that dopamine is produced in several areas of the brain, including the substantia nigra and the ventral tegmental area. Dopamine is also a catecholamine neurohormone that is released by the hypothalamus and involved in various neurological diseases. Particularly, the dysfunction of the dopamine neurotransmitter system leads to Parkinsonian syndrome, schizophrenia, drug addiction, clinical depression and the like. It is known that dopamine is synthesized by mesencephalic neurons in the substantia nigra and the ventral tegmental area, and dopaminergic neurons project from the substantia nigra and the ventral tegmental area to the striatum, the cerebral cortex, the limbic system and the like, and thus are involved in various physiological functions, including motor function, reward circuit response, and the regulation of hormone secretion from the hypothalamus to the pituitary body.

Dopamine is available as an intravenous medication acting on the sympathetic nervous system, producing effects such as increased heart rate and blood pressure. However, because dopamine cannot cross the blood-brain barrier, dopamine given as a drug does not directly affect the central nervous system.

Abnormality in the regulation of dopamine release, for example, the excessive or active release of dopamine, leads to manic depressive illness or schizophrenia, and a decrease in dopamine release leads to clinical depression. Further, damage to neurons that produce dopamine causes motor disturbance, leading to Parkinson's disease. Nicotine absorbed by smoking activates dopamine to give a pleasant sensation. Also, a hallucination or pleasure caused by a narcotic is obtained by the stimulation and activation of dopamine release. Thus, it is important for the treatment of diseases to control the function of dopamine or to inhibit damage to dopaminergic neurons and maintain the growth thereof.

Dopamine functions by its binding to dopamine receptor that is a membrane receptor. It is known that the dopamine receptor binds to G-proteins (GTP-binding-proteins) to activate secondary signal transmitters or activate or inhibit a specific signaling system, leading to physiological responses. The dopamine receptors known to date include five subtypes. According to their structures and pharmacological properties, the dopamine receptors can be classified into D1, D5, a D1-like receptor (hereinafter referred to as D1R) group that activates adenylate cyclase to promote intracellular cAMP formation, D3, D4, and a D2-like receptor (D2R) group that inhibits cAMP formation, contrary to D1R. Because the mechanism of action of D2R is not yet clear, studies have been actively conducted to elucidate the mechanism of action of D2R using knockout mouse models by various molecular or cellular biological methods.

As is known in the art, activation of D2R phosphorylates ERK (extracellular signal-regulated kinases), and in this phosphorylation process, EGF (epidermal growth factor) binds to EGFR (epidermal growth factor receptor) to induce the differentiation of neural progenitor cells into dopaminergic neurons through the MAPK pathway by RAS and RAF. However, the mechanism in which D2R activates EGF has not yet been established.

Accordingly, the present inventors have made extensive efforts to establish the D2R mechanism of dopamine and develop the novel use of neuronal differentiation technology based on this mechanism, and as a result, have found that ADAM17 and ADAM10 are involved in the promotion of differentiation of dopaminergic neurons in the midbrain region, thereby completing the present invention.

### DISCLOSURE OF INVENTION

### Technical Problem

It is an object of the present invention to provide a method for regulating the differentiation of neural stem cells or neural progenitor cells into dopaminergic neural cells, the method comprising increasing or inhibiting the activity of ADAM17 and/or ADAM10 in neural stem cells or neural progenitor cells.

Another object of the present invention is to provide the use of an activator or inhibitor of ADAM17 and/or ADAM10.

### Technical Solution

To achieve the above objects, the present invention provides a method for regulating the differentiation of neural stem cells or neural progenitor cells into dopaminergic neural cells, the method comprising increasing or inhibiting the activity of ADAM17 and/or ADAM10 in neural stem cells or neural progenitor cells.

The present invention also provides a composition for promoting the differentiation of neural stem cells or neural progenitor cells into dopaminergic neurons, the composition containing an activator of ADAM17 and/or ADAM10 as an active ingredient.

The present invention also provides a composition for treating a neurological disease caused by the death of dopaminergic neural cells, the composition containing an activator of ADAM17 and/or ADAM10 as an active ingredient.

The present invention also provides a composition for treating a tumor, the composition containing an inhibitor of ADAM17 and/or ADAM10 as an active ingredient.

The present invention provides an ADAM17 siRNA having a nucleotide sequence of SEQ ID NO: 1.

The present invention also provides a method for screening an agent for treating a neurological disease caused by the death of dopaminergic neural cells, the method comprising the steps of: (a) culturing neural stem cells or neural progenitor cells in the presence of a candidate that activates ADAM17 and/or ADAM10, or treating neural stem cells or neural progenitor cells with a candidate that activates ADAM17 and/or ADAM10; and (b) selecting the candidate that increases the expression or activity of ADAM17 and/or ADAM10 in the cultured or treated neural stem cells or neural progenitor cells as the agent for treating a neurological disease caused by the death of dopaminergic neural cells.

The present invention also provides a method for screening an agent for treating a tumor, the method comprising the steps of: (a) culturing neural stem cells or neural progenitor cells in the presence of a candidate that inhibits the activity of ADAM17 and/or ADAM10, or treating neural stem cells or neural progenitor cells with a candidate that inhibits the activity of ADAM17 and/or ADAM10; and (b) selecting the candidate that reduces the expression or activity of ADAM17 and/or ADAM10 in the cultured or treated neural stem cells or neural progenitor cells as the tumor-treating agent.

Other features and embodiments of the present invention will be more apparent from the following detailed descriptions and the appended claims.

### BRIEF DESCRIPTION OF THE DRAWINGS

FIG. 1 shows the results which indicate that the development of midbrain neurons derived from normal mice and D2R−/− mice into dopaminergic neural cells (TH-positive cells) is induced by EGF (A: immunostaining of midbrain neurons and control; B: the number of TH-positive cells; C: neurite length; D: neurite number; scale bar: 100 um;*, p<0.05; **, p<0.01; ***, p<0.001 control vs. drug-treated group; †, p<0.05; ††, p<0.01 normal mice vs. D2R−/− mice).

FIG. 2 shows the results which indicate that the development of midbrain neurons derived from normal mice and D2R−/− mice into dopaminergic neural cells (TH-positive cells) is induced by a D2R agonist (A: immunostaining of midbrain neurons and control; B: the number of TH-positive cells; C: neurite length; D: neurite number; scale bar: 100 um;*, p<0.05; **, p<0.01; ***, p<0.001 control vs. drug-treated group; †, p<0.05; ††, p<0.01 normal mice vs. D2R−/− mice).

FIG. 3 shows the results which indicate that the phosphorylation of ERK in midbrain neurons derived from normal mice and D2R−/− mice is induced by EGF (A: treated with EGF, quinpirole and haloperidol; B: treated with a combination of EGF, quinpirole and AG1478; *, p<0.05; p<0.01; *** p<0.001 control vs. drug-treated group; †, p<0.05; †††, p<0.001 normal mice vs. D2R−/− mice).

FIG. 4 shows the results which indicate that D2R and metalloprotease are involved in the phosphorylation of ERK in midbrain neurons derived from normal mice and D2R−/− mice (A: treated with a combination of EGF and GM6001; B: treated with a combination of quinpirole and GM6001; ***, p<0.001 control vs. drug-treated group; †, p<0.05; †††, p<0.001 normal mice vs. D2R−/− mice).

FIG. 5 shows the results which indicate that the results which indicate that the development of midbrain neurons derived from normal mice and D2R−/− mice into dopaminergic neural cells (TH-positive cells) is induced by D2R and metalloprotease (A: immunostaining of midbrain neurons and control; B: the number of TH-positive cells; C: neurite length; D: neurite number; scale bar: 100 um; *, p<0.05; **, p<0.01; *** p<0.001 control vs. drug-treated group; †, p<0.05; ††, p<0.01; †††, p<0.001 normal mice vs. D2R−/− mice).

FIG. 6 is a set of photographs showing the expression patterns of ADAM10 and ADAM17 in TH-positive cells in fetal stages SN and VTA (A: ADAM10; B: ADAM17).

FIG. 7 shows the results which indicate that the phosphorylation of ERK by D2R in midbrain neurons derived from normal mice and D2R−/− mice and the development of dopaminergic neural cells (TH-positive cells) by the phosphorylation of ERK are attributable to ADAM17 (A: immunostaining of midbrain neurons and control; B: the number of TH-positive cells; C: neurite length; D: neurite number; scale bar: 100 um; *, p<0.05; **, p<0.01; ***, p<0.001 control vs. drug-treated group; †, p<0.05; ††, p<0.01; †††, p<0.001 normal mice vs. D2R−/− mice).

FIG. 8 shows the results which indicate that the phosphorylation of ERK by D2R in midbrain neural cells derived from normal mice and D2R−/− mice and the development of dopaminergic neurons (TH-positive cells) by the phosphorylation of ERK are attributable to ADAM10 (A: immunostaining of midbrain neurons and control; B: the number of TH-positive cells; C: neurite length; D: neurite number; scale bar: 100 um; *, p<0.05; **, p<0.01; ***, p<0.001 control vs. drug-treated group; †, p<0.05; ††, p<0.01; †††, p<0.001 normal mice vs. D2R−/− mice).

FIG. 9 is a schematic diagram showing the mechanism of dopaminergic neural cells differentiation caused by ERK phosphorylation mediated by D2R and ADAM17 and/or ADAM10 (D2R: dopamine D2 receptor; ADAM: a disintegrin and metalloprotease; HB-EGF: heparin-binding EGF-like growth factor; EGF: epidermal growth factor; EGFR: epidermal growth factor receptor; ERK: extracellular signal-regulated kinase).

### DETAILED DESCRIPTION OF THE INVENTION

Unless defined otherwise, all technical and scientific terms used herein have the same meaning as commonly understood by one of ordinary skill in the art to which the invention pertains. Generally, the nomenclature used herein and the experiment methods which will be described hereinafter are those well known and commonly employed in the art.

In one aspect, the present invention is directed to a method for regulating the differentiation of neural stem cells or neural progenitor cells into dopaminergic neural cells, the method comprising increasing or inhibiting the activity of ADAM17 and/or ADAM10 in neural stem cells or neural progenitor cells.

In the present invention, it was shown that the phosphorylation of extracellular signal-regulated kinases (ERKs) by dopamine D2 receptor and epidermal growth factor (EGF) is a major mechanism of differentiation into dopaminergic neural cells. Particularly, it was shown that ADAM17 and/or ADAM10 are/is involved in the activation of EGF.

In the present invention, the neural stem cells or neural progenitor cells may be midbrain neural stem cells or midbrain neural progenitor cells.

In the method of the present invention, the activity of ADAM17 may be increased using one or more selected from the group consisting of 12-HPETE (12-hydroperoxy-5Z,8Z,10E,14Z-eicosatetraenoic acid), bortezomib (Millennium Pharmaceuticals, USA), Furin, GM-CSF, N-formyl-L-methionyl-phenylalanine, and PMA (phorbol 12-myristate-13-acetate), but is not limited thereto.

In the present invention, the activity of ADAM17 may be inhibited using one or more selected from the group consisting of TAPI-1 (C₂₆H₃₇N₅O₅; Santa Cruze, USA), TAPI-2 (C₁₉H₃₇N₅O₅; Santa Cruze, USA), GW3333 (C₂₂H₃₆N₄O₄), GW280264X (hydroxamate), siRNA, and antisense RNA, but is not limited thereto.

In the present invention, the activity of ADAM10 may be increased using one or more selected from the group consisting of 5α-dihydrotestosterone, donepezil (Pfizer, USA), EGF (epidermal growth factor), PMA (phorbol-12 myristate 13-acetate), and thyrotropin, but is not limited thereto.

Also, the activity of ADAM10 may be inhibited using one or more selected from the group consisting of TAPI-1 (C₂₆H₃₇N₅O₅; Santa Cruze, USA), TAPI-2 (C₁₉H₃₇N₅O₅; Santa Cruze, USA), GI254023X (((2R,3S)-3-(formyl-hydroxyamino)-2-(3-phenyl-1-propyl)butanoic acid)[(1S)-2,2-dimethyl-1-methylcarbamoyl-1-propyl]amide), GM6001 ((2S)—N4-hydroxy-N1-[(1S)-1-(1H-indol-3-ylmethyl)-2-(methylamino)-2-oxoethyl]-2-isobutylsuccinamide), GW280264 (C₂₈H₄₁N₅O₆S), atorvastatin (Pfizer, USA), AEBSF (4-(2-Aminoethyl)benzenesulfonyl fluoride hydrochloride), CMK(decanoyl-RVKR-chloromethylketone), siRNA, and antisense RNA, but is not limited thereto.

In the present invention, the method of regulating the differentiation of neural stem cells or neural progenitor cells into dopaminergic neural cells by the activation or inhibition of ADAM17 and/or ADAM10 may be applied in vitro or in vivo.

As used herein, the term “stem cells” or “progenitor cells” generally refers to undifferentiated cells that are in a stage before differentiation into each type of cells constituting tissue. Stem cells have the ability to divide and also have the capability to differentiate into a specific type of cells when a differentiation stimulus is applied thereto. Also, stem cells have plasticity, which means that they differentiate into various cells having different properties depending on an environment or a stimulus. In addition, stem cells can be classified according to the origin into embryonic stem cells and adult stem cells.

As used herein, the term “neurons” or “neural cells” refers to the cells of the nervous system and may be used interchangeably with the term “neurons” or “neuronal cells”.

As used herein, the term “differentiation” refers to a phenomenon in which the structure or function of cells is specialized during the division, proliferation and growth thereof, that is, the morphology or function of cells or tissues of organisms changes to perform their tasks. Cellular differentiation that is a final stage resulting in the specialization of cells in the developmental process is a phenomenon in which genes in cells are expressed in different manners because their different activities, and as a result, the cells have structurally and functionally completely different characteristics.

As used herein, the term “treatment” means an approach for obtaining beneficial or desired clinical results. For the purpose of the present invention, beneficial or desired clinical results include, but are not limited to, alleviation or amelioration of symptoms or conditions, diminishment of extent of disease, inhibition of aggravation, delay or slowing of disease progression, amelioration or palliation of the disease state, and remission. The term can also mean prolonging survival as compared to expected survival if not receiving treatment. “Treatment” refers to therapeutic treatment, including prophylactic or preventative measures. The treatment encompasses the treatment of a disorder that is prevented, as well as the treatment of an already developed disorder.

As used herein, the term “dopaminergic neural cells” refers to neurons which express tyrosine hydrozylase (TH), are located specifically in the substantia nigra of the midbrain, and stimulate the striatum, the limbic system and neocortex in vivo to regulate postural, movement, and reward-related behavior. Particularly, dopaminergic neural cells should show midbrain characteristics in order to function in vivo.

In another aspect, the present invention is directed to a composition for promoting the differentiation of neural stem cells or neural progenitor cells into dopaminergic neural cells, the composition containing an activator of ADAM17 and/or ADAM10 as an active ingredient.

The present invention is also directed to the use of an activator of ADAM17 and/or ADAM10 for promoting the differentiation of neural stem cells or neural progenitor cells into dopaminergic neural cells.

The present invention is also directed to a method for promoting the differentiation of neural stem cells or neural progenitor cells into dopaminergic neural cells, the method comprising administering an activator of ADAM17 and/or ADAM10.

ADAM17, a member of the ADAM (a disintegrin and a metalloproteinase) family is known as CD156b, cSVP, MGC71942, or TACE (tumor necrosis factor converting enzyme) and has a sequence homology of 30% to ADAM10 and the same α-secretase activity as that of ADAM10.

In the composition for promoting differentiation according to the present invention, the activator of ADAM17 may be one or more selected from the group consisting of 12-HPETE (12-hydroperoxy-5Z,8Z,10E,14Z-eicosatetraenoic acid), bortezomib (Millennium Pharmaceuticals, USA), Furin, GM-CSF, N-formyl-L-methionyl-phenylalanine, and PMA (phorbol 12-myristate-13-acetate), but is not limited thereto.

In the composition for promoting differentiation according to the present invention, the activator of ADAM10 may be one or more selected from the group consisting of 5α-dihydrotestosterone, donepezil (Pfizer, USA), EGF (epidermal growth factor), PMA (phorbol-12 myristate 13-acetate), and thyrotropin, but is not limited thereto.

In the present invention, the method of regulating the differentiation of neural stem cells or neural progenitor cells into dopaminergic neural cells by the activation or inhibition of ADAM17 and/or ADAM10 may be applied in vitro or in vivo.

In still another aspect, the present invention is directed to a composition for treating a neurological disease caused by the death of dopaminergic neural cells, the composition containing an activator of ADAM17 and/or ADAM10 as an active ingredient.

The present invention is also directed to the use of an activator of ADAM17 and/or ADAM10 for preventing or treating a cerebral nervous disease caused by the death of dopaminergic neural cells.

The present invention is also directed to a method for preventing or treating a neurological disease caused by the death of dopaminergic neural cells, the method comprising administering an activator of ADAM17 and/or ADAM10.

In the present invention, the “neurological diseases” include diseases occurring in the brain or the nervous system due to dysfunction or abnormality in the nervous system, including neuralgia, arthritis, headache, schizophrenia, epilepsy, stroke, insomnia, dementia, depression, dyskinesia, dementia with Lewy bodies, Huntington's disease, Tourette syndrome, anxiety, learning and memory impairment, and neurodegenerative diseases. Preferably, the neurological diseases are Parkinson's disease and Alzheimer disease, but are not limited thereto.

In the present invention, the “activator” is an agonist that increases the activity of ADAM17 or ADAM10. Preferably, it may be applied for the treatment of a brain disease caused by the death of dopaminergic neural cells.

The activator of ADAM17 may be one or more selected from the group consisting of bortezomib (Millennium Pharmaceuticals, USA), Furin, and GM-CSF, but is not limited thereto.

The activator of ADAM10 may be one or more selected from the group consisting of 5α-dihydrotestosterone, donepezil (Pfizer, USA), EGF (epidermal growth factor), and thyrotropin, but is not limited thereto.

In yet another aspect, the present invention is directed to a composition for treating a tumor, the composition containing an inhibitor of ADAM17 and/or ADAM10 as an active ingredient.

The present invention is also directed to the use of an inhibitor of ADAM17 and/or ADAM10 for preventing or treating a tumor.

The present invention is also directed to a method for preventing and treating a tumor, the method comprising administering an inhibitor of ADAM17 and/or ADAM10.

In the present invention, the inhibitor is an antagonist that inhibits the activity of ADAM17 or ADAM10.

In the present invention, the tumor may be selected from the group consisting of leukemia, lymphoma, glioma, breast cancer, liver cancer, colorectal cancer, and kidney cancer, but is not limited thereto.

The inhibitor of ADAM17 may be one or more selected from the group consisting of siRNA and antisense RNA, but is not limited thereto.

The inhibitor of ADAM10 may be one or more selected from the group consisting of atorvastatin (Pfizer, USA), siRNA, and antisense RNA, but is not limited thereto.

In a further aspect, the present invention is directed to an ADAM17 siRNA having a nucleotide sequence of SEQ ID NO: 1.

The siRNA or antisense RNA molecule that inhibits the expression of ADAM17 or ADAM10 according to the present invention may have a short nucleotide sequence (e.g., about 5-15 nucleotides) inserted between the self-complementary sense and antisense strands. Particularly, the siRNA molecule formed by the expression of the nucleotide sequence forms a hairpin structure via intramolecular hybridization, resulting in a stem-and-loop structure overall. The stem-and-loop structure is processed in vitro or in vivo to produce an active siRNA molecule capable of mediating RNAi. When siRNA is introduced into cells, the mRNA level of ADAM17 decreases, and thus the activity of ADAM17 decreases.

siRNA can be introduced into cells using a shRNA molecule, and the shRNA construct encodes a stem-loop RNA. After introduction into cells, the step-loop RNA is processed into a double stranded RNA compound, the sequence of which corresponds to the stem of the original RNA molecule. The double stranded RNA can be prepared according to any method known in the art.

For in vivo administration of siRNA or antisense RNA, shRNA or antisense RNA can be inserted into a plasmid to prepare AAV vector, retrovirus vector, particularly lentivirus vector or adenovirus vector, which may be administered in vivo. The vector may be administered by different suitable routes including intravenous route or local injection including intramuscular route, direct injection into subcutaneous tissue or other targeted tissue chosen according to usual practice.

The route of administration of siRNA or antisense RNA varies from local, direct delivery to systemic intravenous administration. The advantage of local delivery is that the doses of siRNA required for efficacy are substantially low since the molecules are injected into or near the target tissue. Local administration also allows for focused delivery of siRNA. For such direct delivery, naked siRNA can be used. “Naked siRNA” refers to delivery of siRNA (unmodified or modified) in saline or other simple excipients such as 5% dextrose. The ease of formulation and administration of such molecules makes this an attractive therapeutic approach. Naked DNA can also be formulated into lipids especially liposomes.

Systemic application of siRNA or antisense RNA is often less invasive and, more importantly, not limited to tissues which are sufficiently accessible from outside. For systemic delivery, siRNA can be formulated with cholesterol conjugate, liposomes or polymer-based nanoparticles. Liposomes are traditionally used in order to provide increased pharmacokinetics properties and/or decreased toxicity profiles. They allow significant and repeated success in vivo delivery. Currently, the use of lipid-based formulations of systemic delivery of siRNA, especially to hepatocytes, appears to represent one of the most promising near-term opportunities for development of RNAi therapeutics. Formulation with polymers such as dynamic polyconjugates (for example, coupled to N-acetylglucosamine for hepatocytes targeting) and cyclodextrin-based nanoparticles allow both targeted delivery and endosomal escape mechanisms. Others polymers such as atelocollagen and chitosan allow therapeutic effects on subcutaneous tumor xenografts as well as on bone metastases.

siRNA can also be directly conjugated with a molecular entity designed to help targeted delivery. Given the nature of the siRNA duplex, the presence of the inactive or sense stand makes for an ideal site for conjugation. Examples of conjugates are lipophilic conjugates such as cholesterol, or aptamer-based conjugates.

Cationic peptides and proteins are also used to form complexes with the negatively charged phosphate backbone of the siRNA duplex.

The composition for treatment according to the present invention may further comprise a suitable carrier, excipient or diluent that is generally used in the preparation of therapeutic compositions, in addition to an activator or inhibitor of ADAM17 and/or ADAM10, an siRNA or an antisense RNA, which is a therapeutically active ingredient.

Examples of carriers, excipients and diluents that can be contained in the composition for treatment according to the present invention include lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinylpyrrolidone, water, methylhydroxybenzoate, propylhydroxybenzoate, talc, magnesium stearate, and mineral oil.

The composition for treatment according to the present invention can be formulated according to a conventional method. For example, it may be formulated in the form of powders, granules, tablets, capsules, suspensions, emulsions, syrups, and aerosols for oral administration, agents for external applications, suppositories, and sterile injection solutions.

A composition for treatment according to the present invention is formulated using diluents or excipients, such as fillers, extenders, binders, wetting agents, disintegrants or surfactants, which are commonly used. Solid Formulations for oral administration include tablets, pills, powders, granules, capsules, etc. Such solid Formulations are prepared by mixing the composition of present invention with at least one excipient, such as starch, calcium carbonate, sucrose, lactose, or gelatin.

In addition to simple expedients, lubricants such as magnesium stearate, talc, etc. may also be added. Liquid Formulations for oral administration, such as suspensions, internal solutions, emulsions, syrups, etc., may include simple diluents which are commonly used, e.g., water and liquid paraffin, as well as various excipients, e.g., wetting agents, sweeteners, aromatics, preservatives, etc.

Formulations for parenteral administration include sterilized aqueous solutions, non-aqueous solvents, suspensions, emulsions, lyophilized agents, suppositories, etc. Non-aqueous solvents and suspensions may be prepared using propylene glycol, polyethylene glycol, vegetable oils such as olive oil, or injectable esters such as ethyloleate. As a base for suppositories, Witepsol, Macrogol, Tween 61, cacao fat, laurin fat, glycerogelatin, etc. may be used.

The preferred dose of an activator, an inhibitor of ADAM17 and/or ADAM10, siRNA and antisense RNA that can be used in the present invention can be suitably selected depending on the route of administration, the severity of disease, the patient's sex, weight and age, etc.

The composition for treatment of the present invention may be administered by various routes to mammals, including rats, mice, livestock and humans. All routes of administration can be contemplated and include, for example, oral, tissue, rectal, intravenous, nasal cavity, intramuscular, subcutaneous, intrauterine, intrathecal or intracerebrovascular injections.

In a still further aspect, the present invention is directed to a method for screening an agent for treating a neurological disease caused by the death of dopaminergic neural cells, the method comprising the steps of: (a) culturing neural stem cells or neural progenitor cells in the presence of a candidate that activates ADAM17 and/or ADAM10, or treating neural stem cells or neural progenitor cells with a candidate that activates ADAM17 and/or ADAM10; and (b) selecting the candidate that increases the expression or activity of ADAM17 and/or ADAM10 in the cultured or treated neural stem cells or neural progenitor cells as the agent for treating a neurological disease caused by the death of dopaminergic neural cells.

In a yet further aspect, the present invention is directed to a method for screening an agent for treating a tumor, the method comprising the steps of: (a) culturing neural stem cells or neural progenitor cells in the presence of a candidate that inhibits the activity of ADAM17 and/or ADAM10, or treating neural stem cells or neural progenitor cells with a candidate that inhibits the activity of ADAM17 and/or ADAM10; and (b) selecting the candidate that reduces the expression or activity of ADAM17 and/or ADAM10 in the cultured or treated neural stem cells or neural progenitor cells as the tumor-treating agent.

In the present invention, examples of the candidate include, but are not limited to, a mixture of unknown chemical substances or compounds, nucleotides, antisense oligonucleotides, siRNAs (small interference RNAs), cell extracts, cell culture supernatants, microbial products during fermentation, marine organism extracts, plant extracts, purified proteins or crude proteins, and peptides, which increase or inhibit the activity of ADAM17 or ADAM10.

Also, the activation or inhibition of ADAM17 or ADAM10 can be determined by direct or indirect methods, including a change in the expression of ADAM17 or ADAM10, the activation of EGF, and the phosphorylation of ERK, but are not limited thereto.

The change in the expression of ADAM17 or ADAM10 can be measured by various methods known in the art. For example, it can be measured by RT-PCR (Sambrook et al, Molecular Cloning. A Laboratory Manual, 3rd ed. Cold Spring Harbor Press, 2001), Northern blotting (Peter B. Kaufma et al., Molecular and Cellular Methods in Biology and Medicine, 102-108, CRCpress), hybridization using a cDNA microarray (Sambrook et al, Molecular Cloning. A Laboratory Manual, 3rd ed. Cold Spring Harbor Press, 2001), and in situ hybridization (Sambrook et al, Molecular Cloning. A Laboratory Manual, 3rd ed. Cold Spring Harbor Press, 2001).

The change in amount of protein with the change in the expression of ADAM17 or ADAM10 can be measured by various immunoassay methods known in the art, including, but not limited to, radioactive immunoassay, radioactive immunoprecipitation, ELISA (enzyme-linked immunosorbent assay), capture-ELISA, inhibition or competition assay, and sandwich assay. The immunoassay method or the immunostaining method is described in Enzyme Immunoassay, E. T. Maggio, ed., CRC Press, Boca Raton, Fla., 1980; Gaastra, W. Enzymelinked immunosorbent assay (ELISA), in Methods in Molecular Biology, Vol. 1, Walker, J. M. ed., Humana Press, N J, 1984; and Ed Harlow and David Lane, Using Antibodies: A Laboratory Manual, Cold Spring Harbor Laboratory Press, 1999.

As used herein, the term “antisense oligonucleotide” refers to a DNA or RNA or its derivatives having a nucleic acid sequence complementary to the sequence of a specific mRNA. The antisense oligonucleotide acts to inhibit the translation of mRNA into a protein by binding to the complementary sequence in the mRNA. The length of the antisense oligonucleotide is 6 to 100 bases, preferably 8 to 60 bases, and more preferably 10 to 40 bases. The antisense oligonucleotide may be modified at one or more positions of the bases, sugars or backbones in order to have improved effectiveness (De Mesmaeker et al., Curr Opin Struct Biol., 5(3):343-55, 1995). The oligonucleotide backbone may be modified, for example, with phosphorothioates, phosphotriesters, methyl phosphonates, short chain alkyl, cycloalkyl, or short chain heteroatomic or heterocyclic intersugar linkages. Also, the antisense oligonucleotide may contain one or more substituted sugar moieties.

The antisense oligonucleotide may also contain modified bases. Examples of the modified bases include hypoxanthine, 6-methyladenine, 5-methylpyrimidines (especially, 5-methylcytosine), 5-hydroxymethylcytosine (HMC), glycosyl HMC, gentiobiosyl HMC, 2-aminoadenine, 2-thiouracil, 2-thiothymine, 5-bromouracil, 5-hyroxymethyluracil, 8-azaguanine, 7-deazaguanine, N6 (6-aminohexyl) adenine, and 2,6-diaminopurine. In addition, the antisense oligonucleotide of the present invention may be chemically bonded to one or more moieties or conjugates that enhance the activity and cellular uptake of the antisense oligonucleotide. For example, liphophilic moieties include, but are not limited to, a cholesterol moiety, a cholesteryl moiety, cholic acid, a thioether, a thiocholesterol, an aliphatic chain, a phospholipid, a polyamine chain, a polyethylene glycol chain, adamantane acetic acid, a palmityl moiety, an octadecylamine moiety and a hexylamino-carbonyl-oxycholesterol moiety. A method of preparing oligonucleotides including lipid moieties is well known in the art (see U.S. Pat. Nos. 5,138,045, 5,218,105 and 5,459,255). The modified oligonucleotide may have enhanced stability in the presence of nucleases and enhanced binding affinity to target mRNA.

The antisense oligonucleotide may be synthesized in vitro by a conventional method and administered in vivo, or may be synthesized in vivo. A method for synthesizing antisense oligonucleotide in vitro employs RNA polymerase I. A method for synthesizing antisense RNA in vivo involves performing transcription of antisense RNA using a vector containing a multicloning site (MCS) in the opposite direction. Such antisense RNA preferably contains a translation stop codon in its sequence to block translation into a peptide sequence.

### EXAMPLES

Hereinafter, the present invention will be described in further detail with reference to examples. It will be obvious to a person having ordinary skill in the art that these examples are illustrative purposes only and are not to be construed to limit the scope of the present invention.

### Example 1

### Examination of the Ability of EGF to Induce Differentiation into Dopaminergic Neural Cells

(1) Examination of Induction of Differentiation into Dopaminergic Neural Cells by EGF Treatment

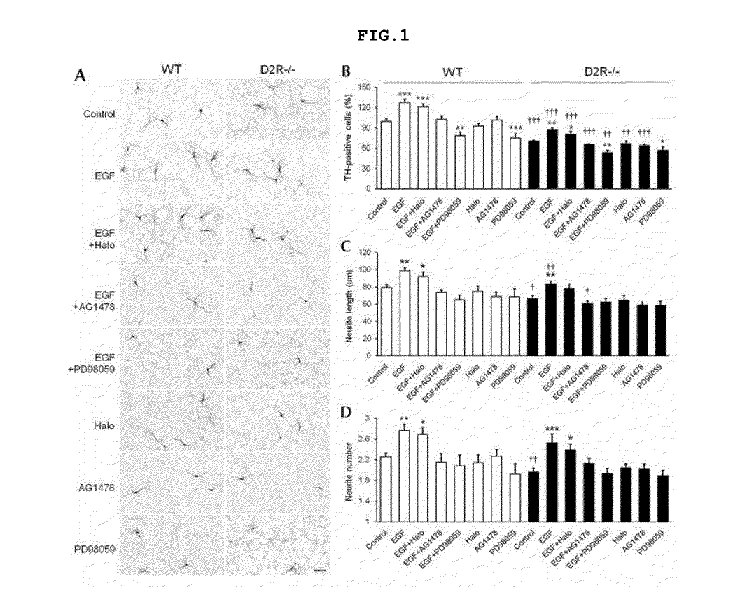

Midbrain neurons derived from normal mice and D2R−/− mice were treated with each of EGF, EGF+haloperidol, EGF+AG1478 and EGF+PD98059, and then stained with TH by an immunostaining technique. Then, the number of TH-positive cells, the length of neurites and the number of neurites in the midbrain neurons were compared between EGF, EGF+haloperidol, EGF+AG1478 and EGF+PD98059 to determine the abilities to induce differentiation into dopaminergic neural cells. As a result, it was shown that the differentiation of dopaminergic neural cells from all the normal mouse and D2R−/− mouse midbrain neurons was promoted by EGF and that this effect was completely blocked by the EGFR inhibitor AG1478 and the MAPK inhibitor PD98059, but was not blocked by the D2R antagonist haloperidol (see FIG. 1).

(2) Examination of the Ability of D2R Agonist to Induce Differentiation into Dopaminergic Neural Cells

Midbrain neurons derived from normal mice and D2R−/− mice were treated with each of quinpirole, EGF plus quinpirole, quinpirole plus AG1478, EGF plus quinpirole plus AG1478, and EGF plus quinpirole plus haloperidol, and then stained with TH by an immunostaining technique. Then, the number of TH-positive cells, the length of neurites and the number of neurites in the midbrain neurons were compared to determine the abilities to induce differentiation into dopaminergic neural cells.

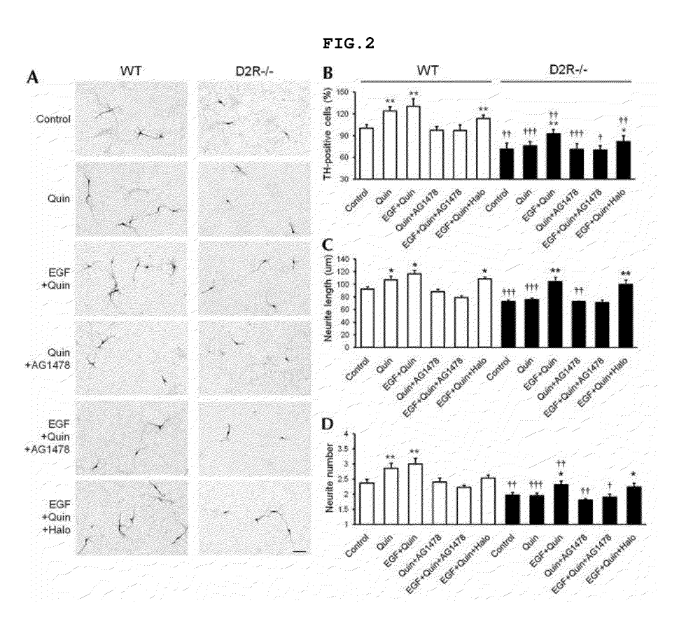

As a result, it was shown that, even when the cells were treated with EGF together with the D2R agonist quinpirole, no synergistic effect on the increase in the number of dopaminergic neural cells in the normal mice was observed, and that the development of dopaminergic neural cells by EGF+quinpirole was not inhibited by haloperidol in both the normal mice and the D2R−/− mice, but was inhibited to the basal level by AG1478 (see FIG. 2).

The above results indicate that D2R is located in a higher signaling cascade compared to EGF and induce the development of dopaminergic neural cells by EGFR.

### Example 2

### Phosphorylation of ERK by EGF

(1) Examination of the Abilities to Phosphorylate ERK by D2R and EGF

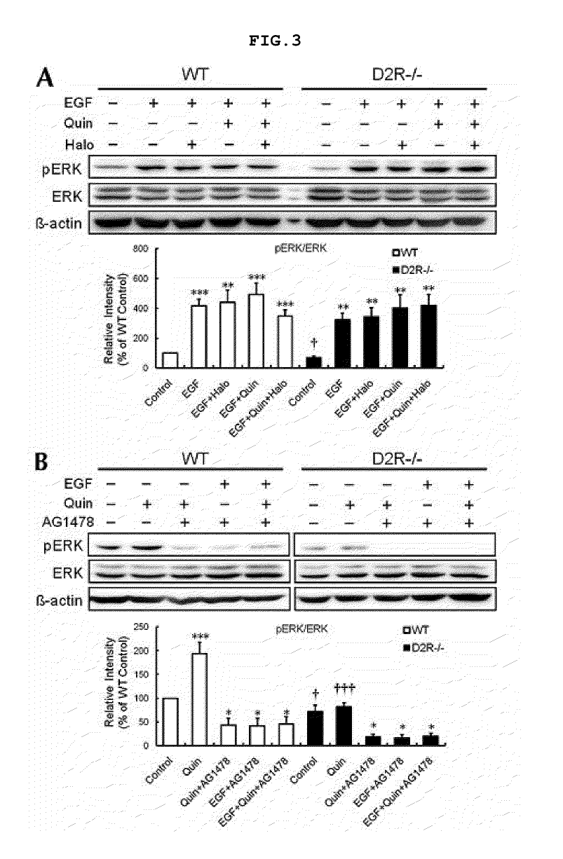

In order to examine whether the development of dopaminergic neural cells by D2R and EGFR is made through the ERK signaling cascade, cells were treated with EGF and quinpirole together with haloperidol and AG1478, and the phosphorylation of ERK in the cells was observed. As a result, it was shown that the phosphorylation of ERK by EGF was increased by 417% compared to that in the control and that this effect was not inhibited by haloperidol. However, it was shown that the phosphorylation of ERK by quinpirole (194%) was inhibited by AG1478 (see FIG. 3).

This suggests that the dopamine D2 receptor promotes the development of dopaminergic neural cells by phosphorylating ERK in an EGFR-dependent manner.

(2) Examination of the Effect of Metalloprotease on D2R-Mediated Activation of EGFR

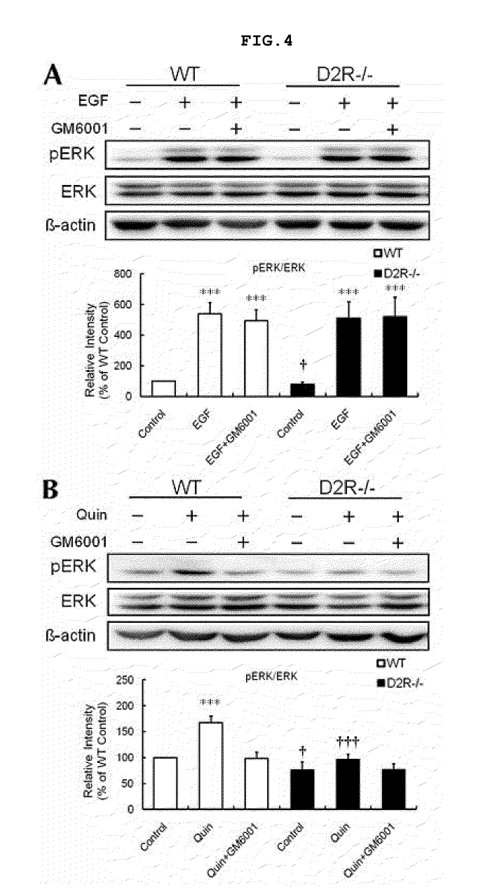

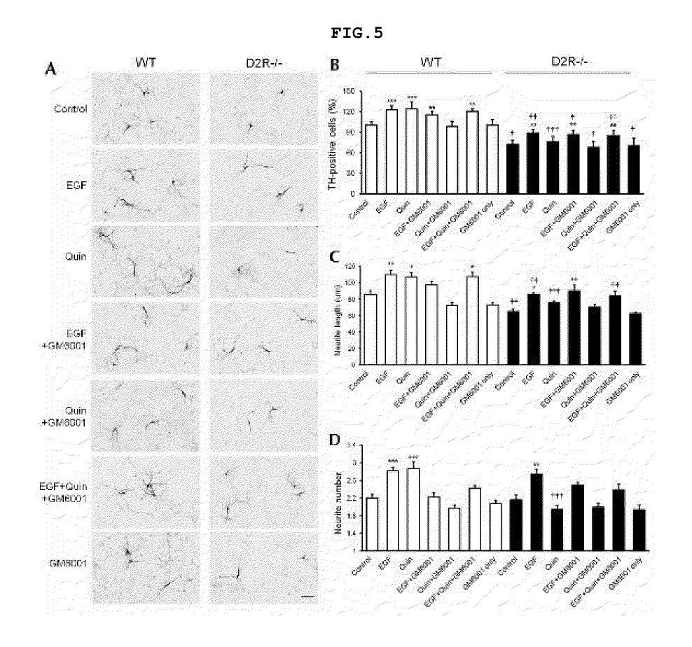

In order to examine whether the development of dopaminergic neural cells by D2R is made through the ERK signaling cascade and whether metalloprotease is involved in the D2R-mediated activation of EGFR, cells were treated with the metalloprotease inhibitor GM6001 together with EGF or quinpirole, and the phosphorylation of ERK in the cells was observed. The phosphorylation of ERK by EGF was not influenced by GM6001, but the phosphorylation of ERK by quinpirole was inhibited to the basal level (see FIG. 4). In addition, it was shown that the effect of EGF on the development of dopaminergic neural cells was not inhibited by GM6001, but the effect of quinpirole was inhibited by GM6001 (see FIG. 5).

### Example 3

### Examination of the Effect of ADAM17 on Phosphorylation of ERK by D2R and EGFR

The present inventors performed immunohistofluorescent staining for ADAM10 and ADAM17, which have been most actively studied in the brain in connection with EGFR, and the present inventors compared the expression pattern of the dopaminergic neural cells-specific protein tyrosine hydroxylase (TH) in the substantia nigra and the ventral tegmental area on 14 days of mouse fetal development.

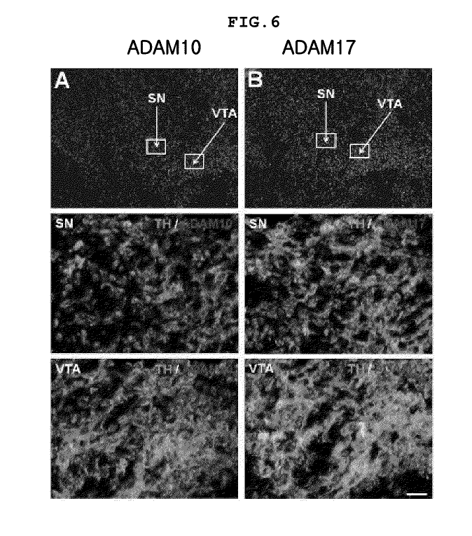

As a result, it was shown that both ADAM10 and ADAM17 were expressed in some TH-positive cells (see FIG. 6).

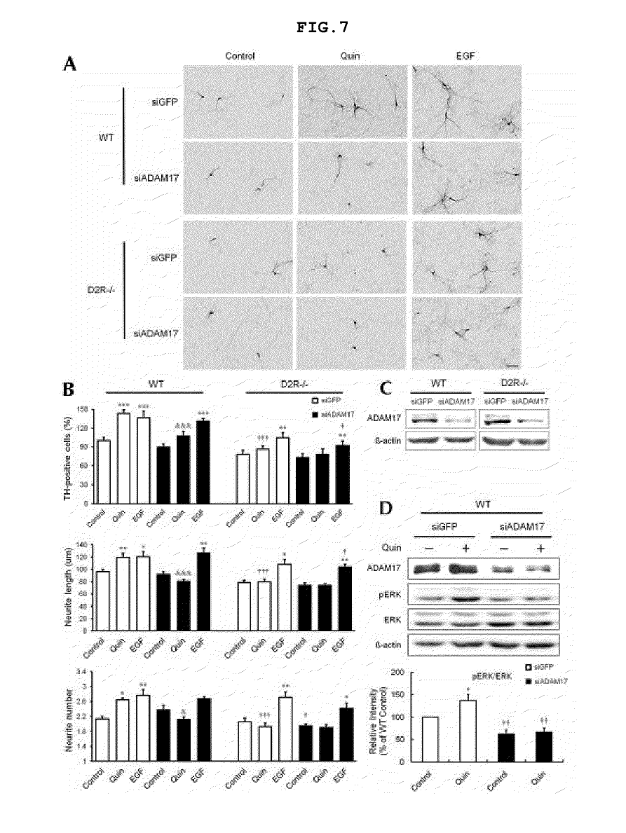

In addition, in order to examine whether ADAM17 is involved in the DR2-mediated activation of EGFR that induces the development of dopaminergic neural cells and phosphorylates ERK, a constructed ADAM17 siRNA (SEQ ID NO: 1; siADAM17) was transfected into primarily cultured midbrain neurons, and then the development of dopaminergic neural cells by quinpirole and EGF and the phosphorylation of ERK by quinpirole were observed. As a result, it was shown that, when glycosylated ADAM17 (100 kDa) was knockdown, the development of dopaminergic neural cells by EGF was not influenced, but the effect of quinpirole was partially reduced (see FIGS. 7A and 7B), and the phosphorylation of ERK by quinpirole was also inhibited (see FIG. 7D). Such results suggest that ADAM17 is involved in the phosphorylation of ERK by the activation of D2R.

siADAM17(antisense):
(SEQ ID: 1)
5′-GGCAGACUUUAGAUGCUUCUUTT-3′

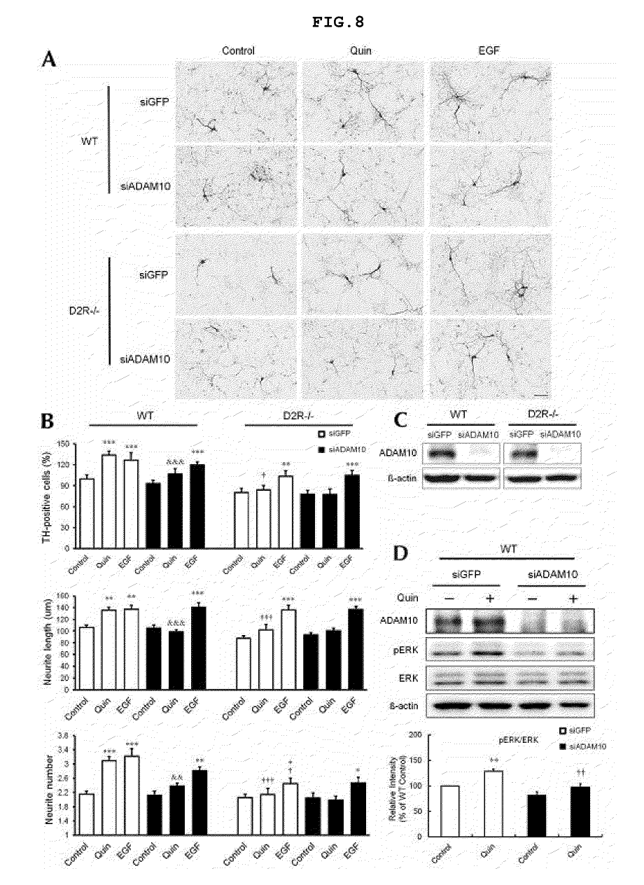

In addition, in order to examine whether ADAM10 is involved in the D2R-mediated activation of EGFR that induces the development of dopaminergic neurons and phosphorylates, a constructed ADAM10 siRNA (SEQ ID NO: 2; siADAM10) was transfected into primarily cultured midbrain neurons, and then the development of dopaminergic neural cells by quinpirole and EGF and the phosphorylation of ERK by quinpirole were observed. As a result, it was shown that, when glycosylated ADAM10 (100 kDa) was knockdown, the development of dopaminergic neural cells by EGF was not influenced, but the effect of quinpirole was partially reduced (see FIGS. 8A and 8B), and the phosphorylation of ERK by quinpirole was also inhibited (see FIG. 8D). Such results suggest that ADAM17 is involved in the phosphorylation of ERK by the activation of D2R.

siADAM10 (antisense):
(SEQ ID: 2)
5′-UCUUCCAUCAAUGACAGACCCTT-3′

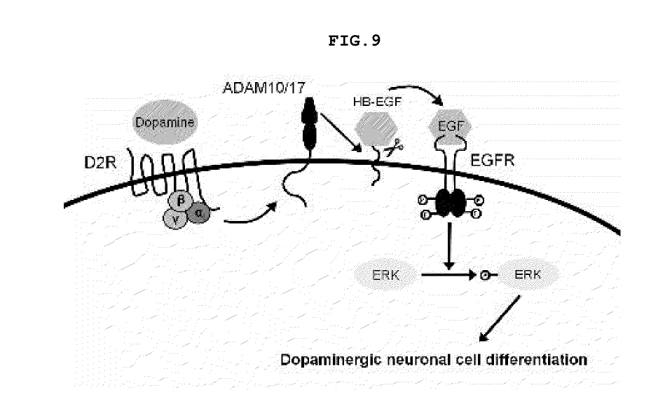

It could be seen that the mechanisms of action of ADAM17 and ADAM10 on the signaling of D2R and EGFR act to dissociate the EGF precursor to regulate the phosphorylation of ERK in a manner dependent on the activation of EGFR to thereby regulate differentiation into dopaminergic neural cells (see FIG. 9).

[Description of Codes]

AG1478 is an inhibitor of EGFR; PD98059 is an inhibitor of MAPK; GM6001 is an inhibitor of metalloprotease; Quinpirole is an agonist of D2R; and Haloperidol is an antagonist of D2R.

### INDUSTRIAL APPLICABILITY

As described above, the method and composition according to the present invention can regulate the activity of ADAM17 and/or ADAM10 in neural stem cells or neural progenitor cells to increase dopaminergic neural cells in the midbrain area, and thereby providing the effect of treating diseases (e.g., Parkinson's disease) induced by the death of dopaminergic neural cells. In addition, the method and composition according to the present invention can inhibit the activity of ADAM17 and/or ADAM10, and thereby improving effect of treating diseases such as tumor. Thus, the present invention is very useful.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. A method for regulating the differentiation of neural stem cells or neural progenitor cells into dopaminergic neural cells, the method comprising increasing or inhibiting the activity of ADAM17 and/or ADAM10 in neural stem cells or neural progenitor cells.

2. The method of claim 1, wherein the activity of ADAM17 is increased using one or more selected from the group consisting of 12-HPETE (12-hydroperoxy-5Z,8Z,10E,14Z-eicosatetraenoic acid), bortezomib (Millennium Pharmaceuticals, USA), Furin, GM-CSF, N-formyl-L-methionyl-phenylalanine, and PMA (phorbol 12-myristate-13-acetate).

3. The method of claim 1, wherein the activity of ADAM17 is inhibited using one or more selected from the group consisting of TAPI-1 (C₂₆H₃₇N₅O₅), TAPI-2 (C₁₉H₃₇N₅O₅), GW3333 (C₂₂H₃₆N₄O₄), GW280264X (hydroxamate), siRNA, and antisense RNA.

4. The method of claim 1, wherein the activity of ADAM10 is increased using one or more selected from the group consisting of 5α-dihydrotestosterone, donepezil, EGF (epidermal growth factor), PMA (phorbol-12 myristate 13-acetate), and thyrotropin.

5. The method of claim 1, wherein the activity of ADAM10 is inhibited using one or more selected from the group consisting of TAPI-1 (C₂₆H₃₇N₅O₅), TAPI-2(C₁₉H₃₇N₅O₅), GI254023X (((2R,3S)-3-(formyl-hydroxyamino)-2-(3-phenyl-1-propyl)butanoic acid)[(1S)-2,2-dimethyl-1-methylcarbamoyl-1-propyl]amide), GM6001 ((2S)—N4-hydroxy-N1-[(1S)-1-(1H-indol-3-ylmethyl)-2-(methylamino)-2-oxoethyl]-2-isobutylsuccinamide), GW280264 (C₂₈H₄₁N₅O₆S), atorvastatin, AEBSF (4-(2-Aminoethyl)benzenesulfonyl fluoride hydrochloride), CMK(decanoyl-RVKR-chloromethylketone), siRNA, and antisense RNA.

6. The method of claim 1, wherein the neural stem cells or neural progenitor cells are midbrain neural stem cells or midbrain neural progenitor cells.

7. A method for promoting the differentiation of neural stem cells or neural progenitor cells into dopaminergic neurons, the method comprising administering an activator of ADAM17 and/or ADAM10 as an active ingredient.

8. The method of claim 7, wherein the activator of ADAM17 is one or more selected from the group consisting of 12-HPETE (12-hydroperoxy-5Z,8Z,10E,14Z-eicosatetraenoic acid), bortezomib, Furin, GM-CSF, N-formyl-L-methionyl-phenylalanine, and PMA (phorbol 12-myristate-13-acetate).

9. The method of claim 7, wherein the activator of ADAM10 is one or more selected from the group consisting of 5α-dihydrotestosterone, donepezil, EGF (epidermal growth factor), PMA (phorbol-12 myristate 13-acetate), and thyrotropin.

10.-18. (canceled)

19. A method for screening an agent for treating a neurological disease caused by the death of dopaminergic neural cells, the method comprising the steps of:
(a) culturing neural stem cells or neural progenitor cells in the presence of a candidate that activates ADAM17 and/or ADAM10, or treating neural stem cells or neural progenitor cells with a candidate that activates ADAM17 and/or ADAM10; and
(b) selecting the candidate that increases the expression or activity of ADAM17 and/or ADAM10 in the cultured or treated neural stem cells or neural progenitor cells as the agent for treating a neurological disease caused by the death of dopaminergic neural cells.

20. (canceled)

21. A method for treating a neurological disease caused by the death of dopaminergic neural cells, the comprising administering an activator of ADAM17 and/or ADAM10 as an active ingredient.

22. The method of claim 21, wherein the activator of ADAM17 is one or more selected from the group consisting of bortezomib, Furin, and GM-CSF.

23. The method of claim 21, wherein the activator of ADAM10 is one or more selected from the group consisting of 5α-dihydrotestosterone, donepezil (Pfizer, USA), EGF (epidermal growth factor), PMA (phorbol-12 myristate 13-acetate), and thyrotropin.

24. The method of claim 21, wherein the neurological disease is Parkinson's disease or Alzheimer disease.

25. The method of claim 22, wherein the neurological disease is Parkinson's disease or Alzheimer disease.

26. The method of claim 23, wherein the neurological disease is Parkinson's disease or Alzheimer disease.
